(19) United States
(12) Patent Application Publication
(10) Pub. No.: US 2024/0268734 A1
(43) Pub. Date: Aug. 15, 2024

(54) Mind Watcher Device

(71) Applicant: Kulangara Sivadas, Foothill Ranch, CA (US)

(72) Inventor: Kulangara Sivadas, Foothill Ranch, CA (US)

(21) Appl. No.: 18/644,094

(22) Filed: Apr. 24, 2024

Related U.S. Application Data
(63) Continuation-in-part of application No. 12/931,101, filed on Jan. 24, 2011, now Pat. No. 11,471,091.

(51) Int. Cl. A61B 5/16; A61B 5/00

(52) U.S. Cl. CPC A61B 5/165; A61B 5/486; A61B 5/681; A61B 5/7267; A61B 5/7445; A61B 5/7475

(57) ABSTRACT

A wearable device and associated method to make use of binary mental state transitions, to increase mindfulness of the wearer, is disclosed. It works from a Physiological Response Model of the wearer's binary mental states, which is derived from signal segments corresponding to those states. The device has a minimal-interaction user interface panel and a quick-glance, intuitive infographic display. It also comes with non-invasive, guided sessions for mindful breathing and mindful walking.

### INCORPORATION BY REFERENCE TO RELATED APPLICATION

Any and all priority claims identified in the Application Data Sheet, or any correction thereto, are hereby incorporated by reference under 37 CFR 1.57. This application is a continuation of U.S. application Ser. No. 12/931,101 filed Jan. 24, 2011.

### FIELD OF THE INVENTION

It broadly applies to mental health monitoring and improving systems.

### BACKGROUND OF THE INVENTION

Mental health seekers nowadays have a variety of assistive devices and app subscriptions to choose from. Many wearables like smart watches, smart rings, smart braces, smart head bands, smart pendants and even some smart glasses come with mental health applications. Some well-known companies making wearable hardware are Apple, Google, Samsung, Garmin, Oura, WHOOP, Muse, Mendi, Flowly, FocusCalm, FRENZ, Healium, Lowdown Focus, AttentivU, Empatica, Ultrahuman etc. In this category (known as smart wearables), some collect bio signals from wearer's body, search for distinct patterns in them and alert the wearer when appropriate. These could be classified as active intervention devices. Aside from the native-to-device applications, there're a multitude of third-party apps and services in this category such as Stress Tracker, StressWatch, Stress Monitor, Welltory etc. Some smart wearables go to the extent of following up on the alerts with appropriate wellness sessions (such as by launching a de-stressing audio therapy). And then there are some devices that monitor the wearer's bio signals constantly or for specific time intervals and summarize findings in a report. Many smart wearables work in an ‘open-loop manner’, i.e., without checking bio signals that mark the wearer's mental state. They launch their applications at pre-determined intervals asking the wearer to do breathing exercises, mindfulness activities, reflect and journal current mental state or meditate on calming soundscapes. However some apps log bio signals from their bodies concurrently to analyze and deduce their meanings in brief reports. In many cases, findings deduced from bio signals get rolled up into a score (typically between 1 and 100) variously labeled as “stress management score”, “resilience score”, “readiness score” or “vitality score” for the individual. Across the various apps, these scores tend to focus on three factors: mental stress, physical exertion, and sleep quality of the wearing individual.

Other than smart wearables, there're also smartphone apps that work in a stand-alone manner (not tied to specific hardware platforms or bio signals) to instill the habits of mindfulness, meditation etc. to their users. Some examples are Headspace, Calm, Breathe, Smiling Mind etc. Typically, they send audio instructions to the wearer that help relax the body, turn attention inwards, control breathing etc., while creating a suitable ambience using soundscapes, pictures, or videos.

A core issue limiting the efficacy of these mental wellness trackers using biomarkers is that feelings underlying a ‘labeled’ emotional state (mental states such as sadness, anger, excitement) are subjective to the individual's personal upbringing/background and hence its bio-marker wouldn't be quite unique across a population. It's good news that Apple Health app has a “State of Mind” feature in which the wearer could log their current emotional state using 13 different labels. However, its usability becomes questionable because, the cognitive states underlying each label could be felt differently by each wearer and definitions of emotions exist only inside the ‘head’ of the wearer. Stressfulness, relaxedness, attentiveness, mindfulness etc. are relativistic states that are not felt the same way across the population. Individual variations in bio signal patterns underlying common cognitive states further complicates the task of discerning the states. Compounding this issue is the practical difficulty in distinguishing between certain ‘negative’ and ‘positive’ emotions using bio signals alone. For example, bio signals underlying ‘agitation’ may look very similar to that caused by ‘excitement’. Because of the above reasons, many mental health devices often interrupt a wearer's otherwise pleasant day with false positive, untimely triggers while missing on true negative situations at other times.

Another feature of some mental health devices at issue is their complex user interface provided for interacting with the device. For example, consider the number of steps to be taken to access the “State of Mind” feature in Apple Watch and then browsing the various emotional labels to choose the right one and then logging a statement on your mood. Similar difficulties exist in the use of many smart rings and watches during their “reflect” sessions.

A third issue with products in the market is their lack of ability to let their users define own pivotal moments or emotional states of their lives from perspectives of their recurrence, clarity etc. In the art incorporated by reference in this application (U.S. Pat. No. 11,471,091 Mind Strength Trainer), such methods have been extensively taught. Such abilities play a key role in customizing Machine Learnt (ML) models that power the wearables, to suit individual cognitive behaviors.

A fourth issue addressed by this invention is a lack of on-the-go mindfulness exercises available in the market. For example, most of the currently available breathing apps on smart wearables require the wearer to take his/her mind off the task they are currently engaged in, which causes inconvenience to the wearers. The Breathe app from Apple provides a visual animation that the wearer needs to watch to modulate his/her breathing in sync with the animation. Essentially, that requires the wearer to interrupt his work to pay attention to the animation at least for a few minutes. Similarly, the Moonbird breathing device requires you to hold it in your hand to feel its movements while breathing.

### SUMMARY OF THE INVENTION

Accordingly, the current invention is a wearable mental health tracking & improving device (also referred to as ‘the device’ in the foregoing description) that resolves the issues identified in the previous section.

It's also a device that trains mindfulness using personal life events by extending the concept of mind strength training in an open-loop fashion as depicted in FIG. 9 of the referenced art.

Rather than making a wearer deal with dozens of emotions and their flavors, the device needs the wearer to identify just one kind of emotional transient (also called mental state transition) that recurs in his/her life. The transient's kind (type) will be identified with the nature of initial and final emotional states the weather goes through. Those states are called a binary pair of emotions or mental states. A simple user interface comprising a minimal number of buttons is created on the wearable device to facilitate a one touch acknowledgement of either state. Also, a simple, quick-glance infographic display such as a pie chart (on watch face or widget face as applicable to the device) is created to chronologically and comparingly display dwelling durations in either of emotions belonging to the binary pair.

The invention also comprises calculation of a Hit Score and a Positivity Score on a daily (or any fixed time duration) basis, using the device. It further discloses mindfulness exercises that can run in the background while the wearer is engaged in focus-demanding activities.

Another component of the invention is a Physiological Response Model (PRM) under the hood of the device, capable of identifying a member state of said binary pair of emotional states, by analyzing binary pairs of signal segments collected from the wearer.

### DRAWINGS-FIGURES

FIGS. 1A, 1B, 1C and 1D illustrate designs of binary button touch interfaces on smart wearables that could be used for inputting data pertaining to binary states of mind.

FIGS. 2A and 2B show sample user interfaces on smart wearables for comparingly displaying binary mental states, mental wellness indices etc.

FIG. 3 shows a process flow diagram to build or customize a Physiological Response Model (PRM) suitable to a wearer.

FIG. 4 shows typical steps involved for the wearer to acknowledge or report a mental transition.

FIG. 5 shows typical steps involved for the device in reporting a mental transition.

FIG. 6 depicts a process flow diagram to derive and report graphics and metrics for mental wellness using the device.

FIG. 7 illustrates a method for synchronization of breathing and walking rhythms to form a Mindful Walking Rhythm

### DETAILED DESCRIPTION

Human emotions or mental states can be broadly classified into two categories: the ones you like and the ones you don't. This approach of binary classification could be seen in the referenced prior art, U.S. Pat. No. 11,471,091 Mind Strength Trainer. Brooding and non-brooding states discussed there could be considered as a binary complementing pair. For a person experiencing such binary states, they mostly happen in succession and together they form a transient. For this reason, these binary states could also be viewed as the initial and final states of a person's mind that undergoes a transition between them. Labeling these states in a universal sense is tricky because only the person undergoing the transition would be able to differentiate between his/her feelings reliably and repeatedly. An example is differentiating between happiness, sadness, and a neutral state. If a person feels that recovery from his/her sad state happens to be into a happy state most of the time, “sadness-happiness” makes a good binary pair for that person to label such mental transitions. But if the same person believes that sadness is usually followed by a neutral state of the mind, then “sadness-neutrality” or “sadness-non sadness” would be the appropriate choice to define those transitions. Another worthy example is the “rage-calm” pair of emotions experienced by young adults. Ideally, the device user should be given the freedom to differentiate & label an “engaged-disengaged” pair from an “aroused-non aroused” pair, and that is what this invention provides. There would always be some emotional situations known only to us that could only be appropriately described as “stomach-churning” or “goose-bumpy”. For example, what would you label your mental state when you're about to open your office email every morning? The good news is that, irrespective of what you name it, if you experience a distinctive transition of mental state (such as into a sense of relief) after having skimmed through the inbox, it is a sure candidate for defining a transition (between binary states) in the context of this invention. For simplicity, these binary states would also be referred to as positive and negative in some of the discussions.

The task of differentiating a biomarker of a specific mental state in relationship to markers of its complementary pair (in a scheme of binary classification of emotions) is much easier than looking for each state's absolute markers, from the perspective of signal processing and machine learning. Also it helps that recent advancements in biosensing & alerting technology has made it possible to utilize new kinds of bio signals, and experiment with their combinations with the help of Artificial Intelligence tools. These days, there are wearable EMG (Electromyography), EOG (Electrooculography), pupillometry, ERP (Event Related Potential), HRV (Heart Rate Variability), EDA (Electrodermal activity) and even piloerection (goosebump) sensors available in the market to peek into human emotions. Some of the wearables such as mouth-implant platforms can even be completely hidden from plain sight. Gone are the days when engineers had to rely solely on differentiating between the EEG (Electroencephalogram) components such as θ, from β or γ in pursuit of biomarkers for mental states! Advances are also happening in areas of wearable sensory stimulators. For example, prototypes of mood-altering scents and their microfluidic chip delivery systems have already been developed on lab scale. As for mood-altering visual and auditory stimuli, there's no need to look beyond the millions of ASMR (Autonomous Sensory Meridian Response)—triggering video and audio files floating around on the internet.

Having identified and pre-validated as many mental transitions as possible, the current invention enables its wearer to utilize them as moments of self-reflection and positive recovery, thereby imbibing a habit of mindfulness on-the-go (on-task), without necessarily having to go off-task and step through lengthy protocols (typically comprising sessions of closing the eyes, controlled breathing and listening to soundscapes) as done with the current mental-health wearables. Briefly stating, the current invention is a mental state-transition detector triggering on wearer's choices of emotional transitions and whose triggers could be put to use in multiple ways. For example, if the trigger is configured to drive mood-altering sensual experiences personalized for the wearer (referred to as Mantras in the referenced art U.S. Pat. No. 11,471,091 and the present one) upon a transition detection, the wearer would recover to a relatively “positivity feeling state”. Also, Mantras when repeated, bring back the ‘positive’ emotions they were rewarded for at the first place. Further, sustenance of such a ‘positive’ state vis-a-vis ‘negative’ state (i.e., the complementary pair of the ‘positive’ state in the present context) could be monitored throughout the day and summed up to report a ‘net positivity score’ for the day. The positivity score could be as simple as a 1 to 100 percentage, representing the fraction of positivity minutes (to the total number of minutes) as sensed by the device's bio sensors.

The trigger could also be used to launch ‘noninvasive’ mindfulness sessions so that the wearer doesn't need to disengage in any manner from his/her ongoing tasks. For instance, the device would guide the wearer to pace inhalations and exhalations using continually modulated, musical tones or tactile sensations to change wearer's undesirable breathing pattern (such as a shallow and fast breathing). While tones could be played using speakers (such as ear pods), tactile sensations could be delivered using piezo shakers or electroactive braces. The device could also guide the wearer to synchronize his/her walking pace harmoniously with breathing cycles to have a session of mindful walking if so desired by the wearer to mitigate stress. Walking cues are typically transmitted as discrete, rhythmic pulses (audio, haptic etc.) that either overlay or get modulated by the previously mentioned breath-guiding tones. A person wearing the device could manually synchronize his/her breathing and/or walking with these device-generated rhythms. The device could also automate the synchronization process by adjusting ramp ups or ramp downs in accordance with feedback obtained from sensors that monitor wearer's breathing and walking in real time.

Another use case for the trigger is alerting the wearer of his/her sleep-readiness based on the transitions detected, and sequencing transition-inducing Mantras that would slowly nudge him/her towards going to the bed and make feel comfortable. A use case is the wearer donning the device after dinner and sitting in the family room chatting. The device would monitor the metabolic load the body faces (perhaps from variations in blood sugar level, body temperature), body hydration, humidity level, blood oxygen etc. and dissuade him/her from going for that cheesecake or turning up the room temperature. It would also deter the wearer from going to bed before initial digestion is complete (which takes 2-3 hours after the meal). Similarly, the device would monitor the cognitive loads (perhaps using pupillometry, EOG) faced by the wearer and advise him/her to put the smartphone away or stop talking. Once the wearer is quiet and resting, the device could advise to wait another 15 or so minutes while turning on some ASMR soundscapes if so desired. After a while, when appropriate “sleep-readiness transitions” are picked up, the device would advise the wearer to go to bed and cozy up with pleasant feelings. At this stage the wearer would have the choice to remove the device before falling asleep. All through the process, the device looks for (and facilitates) positive transitions and warns about negative transitions that have might've already happened. A “sleep-readiness score” feature as described above would be most helpful to people having difficulty with falling asleep. Feature of monitoring such readiness scores could be extended to any action the wearer is preparing to face, such as an examination, an interview or a dating appointment.

Obviously, the device could also be used in an ‘open loop’ just for monitoring and historically tracking binary states with or without triggering alerts. This mode of usage would be useful when a wearer wants to build a Physiological Response Model of own body to emotions, environments etc., in order to select at least a pair of binary mental states suiting his/her personality. In this use case before logging a transition event, the device would get the binary states labeled and/or validated by the wearer at each occurrence.

Sensory appeal is very important in the case of choosing immersive Mantras if the device is to be used as a behavioral modifier by the wearer. For example, an auditory stimulus presented to the wearer in outdoor environments may not be felt as dramatic or intimate as they do indoors. Mantras chosen for on-task applications must not interfere with the wearer's engagement. For instance, a whiff of air, cold dew drops, tingling sensations, scent of pine trees or even a ‘deafening silence period’ are good choices for on-task alerting. However, projecting the image of a full moon in 3D rising in a blue sky, practically blocking the entire field of vision of the wearer who happens to be driving a car, is not a good idea.

Nearly as important as choosing a personal Mantra is choosing a personal “recovery gesture” that the wearer must practice to self-reflect each time the Mantra goes off. It could be a thought of gratitude, a smile or a whisper. The more such self-reflection sessions happen, the more mindful the wearer becomes, ensuing fewer emotional swings, and boosting the “net positivity score”.

In the best mode of operation of the device, it relies on a ‘preloaded’ generic Physiological Response Model (PRM) when starting operation on a wearer's body. PRM is a bio-mathematical model of the wearer that can simulate the involuntary human physiological responses to internal and external stimuli. They are generally neural network models that are trained using machine learning and cross-validated to an extent. PRMs could also be transfer functions, differential equations, or algebraic relationships. In the context of the current invention, the PRM is derived from segments of wearer's physiological markers (bio signals) corresponding to a designated set of binary mental states collected during each of their repeat occurrences. A generic PRM typically comprises a neural network trained from a widely accepted set of binary bio marker segments (that are known to represent their underlying binary mental states in a transition), and the corresponding binary responses (a case of positive identification, versus no identification of the transition) of a typical human user in each of those experiences. As the wearer continues using the device, the generic PRM gets modified based on the inputs provided by the wearer and gets customized for the wearer's physiological responses and life situations. It is important that the wearer gets an easily accessible, simple-to-use, user interface to provide inputs to the smart device, which would keep them motivated to try different types of emotional situations.

Perhaps the friendliest device-interface for a user to input any emotional data would be voice or gesture based. However, acquisition of biomarker signals (corresponding to the mental transition) is very time critical, since it requires a precision timestamp for trigger point. Therefore, a haptic interface (a touch input on the interface panel on the device surface) is a preferable choice to implement this. Its simplest implementation would have just one button on the interface panel for the wearer to operate (such as, for triggering the acquisition of transition related biomarker data), provided that there is an unambiguous interpretation for that interaction programmed into the device. For example, the single touch action on the single boolean button described above could also convey to the device, the pre-defined type (label) of mental transition and thereby its known binary component labels.

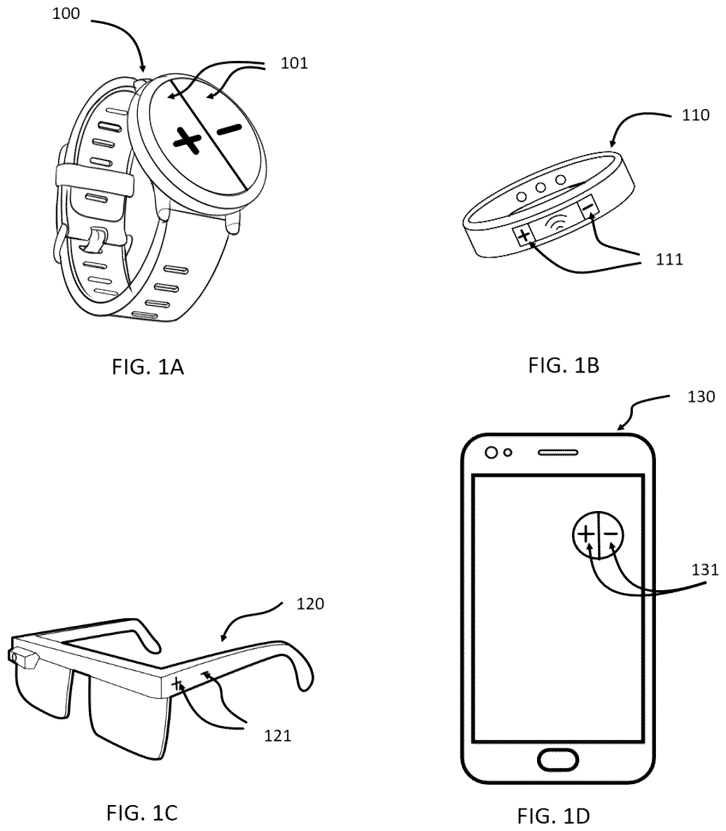

FIGS. 1A, 1B, 1C and 1D illustrate a few design possibilities to facilitate simple and unambiguous inputting of binary responses through the touch interface panel of the device, which happens during dialogs launched by the device in the customization phase. FIG. 1A shows a smart watch with a pair of binary buttons 101 showing on the touchscreen panel. Figure one B is a Smart ring with binary buttons 111 on its side. FIG. 1C depicts a smart glass where the buttons are conveniently positioned on its temple. A Smart phone's touchscreen is shown in FIG. 1D. Binary buttons 131 are implemented on the user interface panel of a widget or app, for fast and easy access. The Plus and the minus marks could indicate a positive and a negative state of mind as defined by the wearer or a yes/no answer in a dialogue with the device. They are marked in pairs 101, 111, 121 and 131 in all the figures. A negative state may indicate anger and a positive could be calm (or a normal state) the wearer. In the case of a smartwatch for example, the wearer will press the plus or minus button to distinguish a particular mood from its binary pair. In another possibility, the wearer could press both the buttons together to indicate a third option such as discard or none of the above. The smartwatch could also simultaneously support multiple definitions of transition labels by having one watch face (interaction panel as implemented by software graphics) dedicated to one transition label. In such cases, the wearer could swipe between watch faces to get to the right one. In a similar way, binary input touch interfaces are shown implemented on Smart rings (FIG. 1B), smart glasses FIG. 1C), and smartphone applications/widgets etc. (FIG. 1D).

In a preferred embodiment of the invention, the touch interface described above is complimented by voice inputting and audio instructing capabilities of the device. In other words, the binary buttons facilitate inputting binary replies (such as yes/no) most quickly and efficiently to auditory queries from the device. Other functions like scrolling down a menu, editing a field etc. would still be available in the display field. Display fields would be available on the device-screens (such as in a smartphone or watch) or would be projected to the view field of a smart glass.

It's desirable to have as many transition-labels as manageable by the wearer stored in the device, so there would be an abundance of their repetitions throughout the wearer's wakeful day, giving many chances for the wearer to reflect upon. It is always easier to start with the most known, familiar, and repetitive transitions that the wearer is aware of.

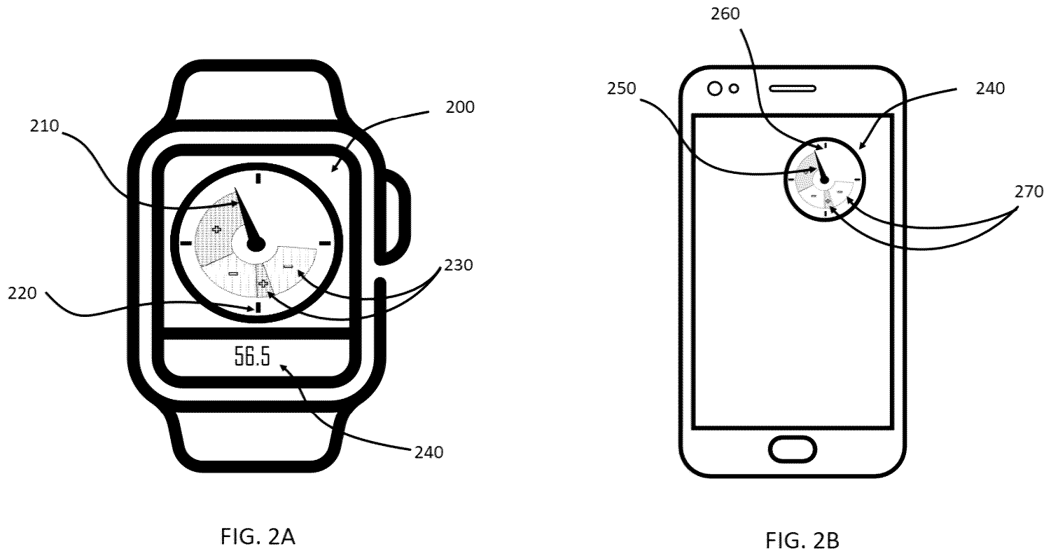

FIGS. 2A and 2B show sample display panels on smart wearables or other devices for comparingly displaying periods of time spent by a person in a pair of binary mental states, in a readily accessible, comprehensive, and quick-glance manner. In FIG. 2A illustration, the dial face 200 has a ‘closed-curve’ time scale such as the circular one shown (220) with a single needle shaped time-hand 210 chronologically moving in the clockwise direction. Range of the time scale could be an hour, a day, a week, or any period chosen by the wearer. While position of the time-hand 210 indicates the time at any given moment, it also indicates one of the two binary states where the wearer's mind is dwelling at that time. Meanwhile, the area (or arc length) swept behind by the time-hand gets painted by a shade or color that represents the corresponding binary state at that time. For example, in FIG. 2A, the total area swept is shown as an annular sector consisting of four segments, each having a different arc length proportional to the four dwelling periods of the wearer's mind that alternated between a “negative” and a “positive” state (those being the labels given to the pair of binary states by the wearer) as indicated by the two types of their shading. Annular Sectors of similar shading represent time durations of similar mental states that the wearer's mind dwelled. Sectors marked as 230 represent a pair of binary states where a negative mental state is followed by a positive mental state. It may be noted that in lieu of using annular sectors, circular sectors could also be used. In the case of using circular sectors, the dial face may look like a pie chart. Either way, sectors displayed in alternating colors/shades according to the binary moods on a chronological scale gives the wearer a very intuitive grasp of his/her mental control in a single glance. FIG. 2B shows a similar chronological display on a smartphone widget that would be easily accessible to the wearer. 270 represents a typical pair of binary mental states that were formed by the time hand 250 when it passed over that area. On the lower portion of the watch face of FIG. 2B, 240 represents the score calculated from the net areas under positive sectors against negative sectors. Implementation of user interfaces on devices with limited work-surface (such as a smart pendant, ring) could be as simple as color changing LEDs or widgets on a mobile phone or smart glass that are remotely (wirelessly) connected to the device. Thus it may be noted that hardware or software components for data acquisition, signal processing, display of analytics etc. need not be co-located physically within the shell of the wearable device.

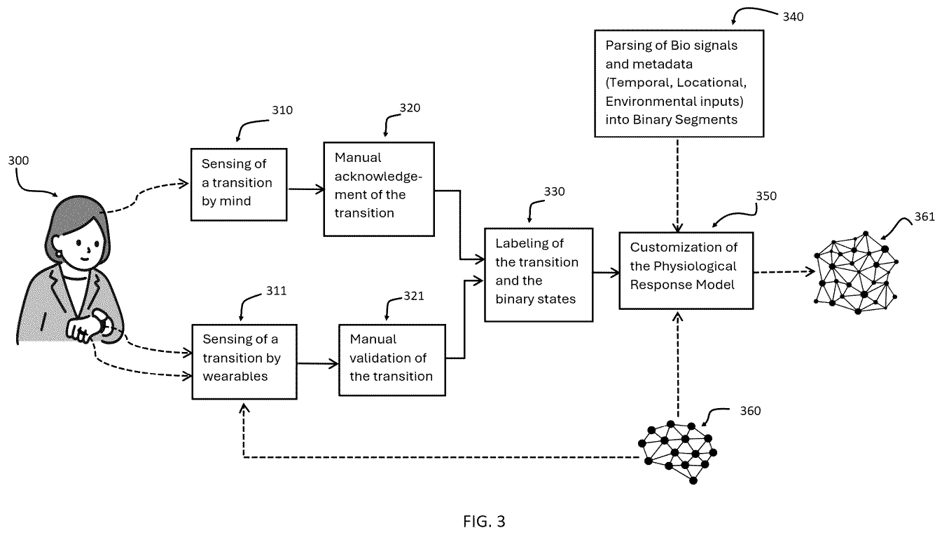

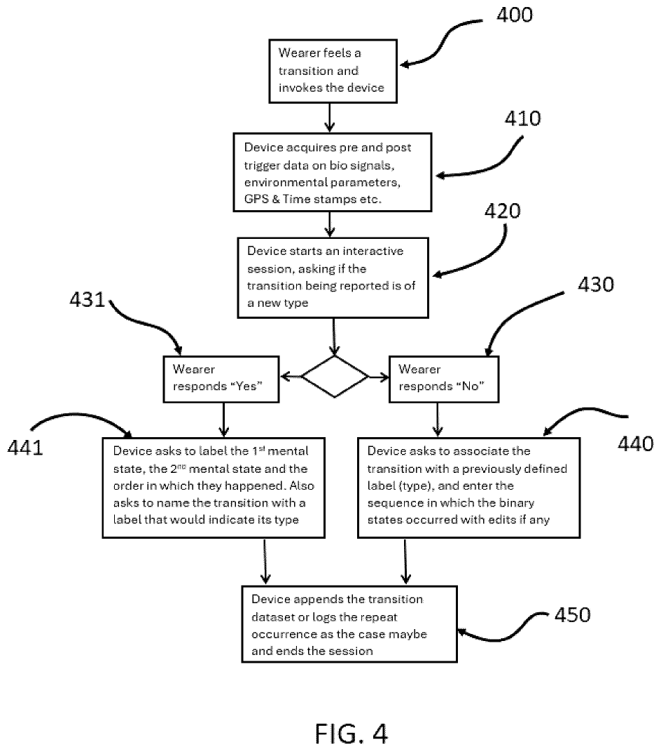

FIG. 3 illustrates a series of steps (blocks) of the operational cycle, that takes to build or modify a physiological response model PRM of binary states, customized for the wearer. Though it runs in the background throughout the operational phase of the device, the process gets more priority during the initial (customization) phase of the device's usage, where it learns from the more frequently occurring emotional transitions and builds a robust model. From FIG. 3, it can be seen that the learning data could come from two branches of the process. In the branch shown at the top, block 310 describes the step of wearer 300 experiencing a mood transition in her mind, and following step 320 to manually acknowledge to the device as immediately as possible. The wearer is preferred to do the acknowledgement using a crisp action (such as a touch on any button on the device's interface panel, a crisp voice command, shaking of the device or through a hand gesture) while this action helps to provide a precise timestamp for triggering data acquisition channels. Acknowledgement needs to be done as soon as the wearer feels (or nearly in tandem with) the transition, in order for the data acquisition channels not to lose buffered data being held in their memory that contains pre trigger history corresponding to the initial parts of the transition. In FIG. 3 the wearer is shown using two smart devices (one on her finger and one on her wrist). It is to imply that simultaneous usage of bio signals of multiple types for sensing the same mental state helps to increase the robustness of the PRM. After acknowledging the transition, the wearer is also required to label the transition, the two states it was comprised of and the order in which the two states occurred (block 330). Steps on manual inputting of a transition and it's binary components has been illustrated in detail in FIG. 4. It may be noted that triggering of data acquisition system to automatically acquire bio signal segments from the wearer's body could also be configured to simultaneously acquire segments of metadata such as environmental signals (weather, pollution, noise level, wearer's voice temperament, wearer's current activity, illness etc.) depending on the sensors available on the wearable device, their timestamps, inflection points, trigger points, geographical and spatial circumstances etc. In most cases, sensors for collecting bio signal segments and metadata segments are analog by choice, and hence capable of streaming continuous signals in a manner that data acquisition channels could digitize them and acquire samples in buffers. However, in cases in some cases such as GPS signals, weather report etc., data segments collected could be digital. Care is taken to acquire a sufficient number of pre- and post-trigger samples so that the acquisition is able to cover the entirety of initial, final and the mid sections of the wearer's mental state transition.

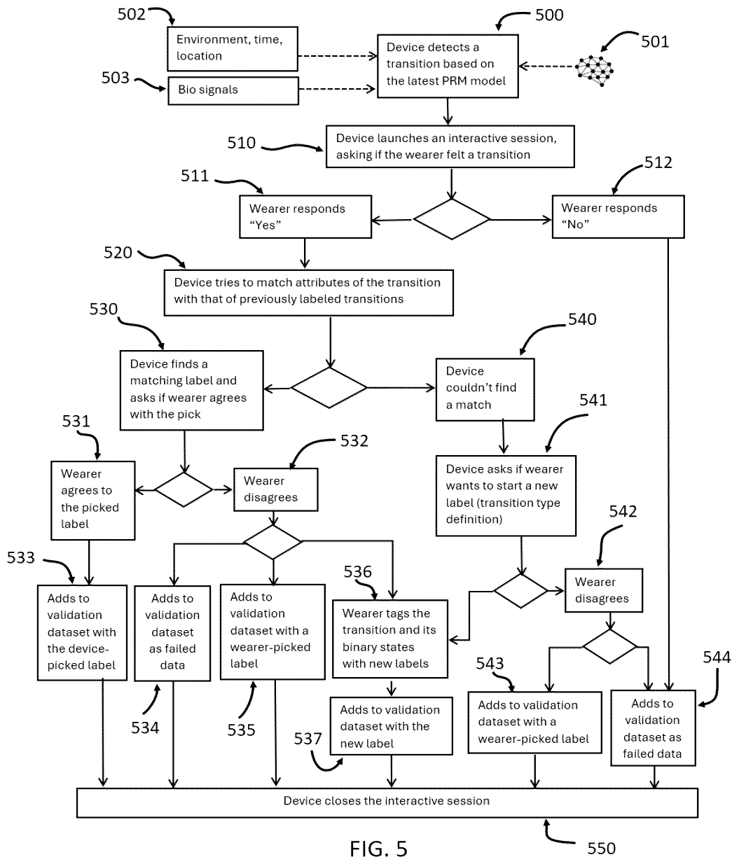

The bottom branch of FIG. 3 depicts the process of automatic transition detection by the sensors embedded in the smart device. Block 311 depicts the step of on board sensors detecting a transition in the wearer's mental state. In the initial stages of operation, the time and amplitude thresholds to detect a transition is automatically set basing off the initial PRM (a generic version) 360 available to the device. Upon a transition detection, the device wants the wearer to validate 321 and label 330 the detection appropriately. Details on this process has been illustrated on FIG. 5. If the weather agrees with the device that there was indeed an occurrence of an emotional transition, he/she is given the opportunity to label the transition and the binary states it is comprised of. On the other hand, the wearer would notify the device of his/her disagreement and the transition would still be added to the validation data set.

During this reinforcement learning phase, the wearer gets the opportunity to know the reliable/more repetitive transitions of his/her life while eliminating the rarely occurring or undetectable ones. Upon each iteration (occurrence of validated transition reporting) transition data segments collected from each bio signal and environmental channels (340) are parsed and fed into process step 350 with timestamps. Thus, transition data in the current context would imply segments of bio signal data and any metadata collected along with bio signals that complements it, such as environmental and behavioral data. Just as in the case of a bio signal segment underlying the wearer's emotional transient gets parsed to extract the binary states, (corresponding to the initial mental state and final mental state), the metadata is also divided into binary sections for processing (340). This is achieved by saving the transition data in a RAM and running a trend analysis to find its distinct parts using appropriate firmware, software etc. At step 350, the newly fed transition data is used to refine the prior version of the PRM 360 to a new version 361, where the machine would assign due weightages to bio signal transients, environmental transients, geologic—spatial—temporal—seasonal—weekly—monthly recurrence factors etc. In one of the possibilities, every iterative loop of FIG. 3 could start with the refined PRM model (361) from the previous loop execution. The refinement iterations could repeat until the device and its wearer would near-unanimously identify the types of transitions (and their binary state components) at each and every one of their occurrences and without false positives or true negatives. The wearer is also given the ability to have multiple transition types defined, labeled, validated, and added to in his/her operational dataset if desired.

FIG. 4 further details the process steps 310, 320 and 330 of FIG. 3 which deals with a wearer-initiated addition of binary mental states and transitions during the customization phase. Blocks 400 and 410 show how the wearer prompts the device to acquire bio signals and associative signals related to a mental transition event that he/she felt immediately before. Wearer's invoking of the device (for acknowledging the transition) could be preferably through a crisp touch, though audio commands or other user interactions are programmed. The invoking generates a trigger signal, which gives the time reference for the device to acquire sufficient lengths of pre-trigger and post-trigger data from its various data acquisition channels. 420 is a step where the device starts gathering the information required to label the transition, its constituent binary states, their succession order, etc. This interactive session could be visual (using a touch sensitive screen) or auditory means (via speakers and microphones). If the wearer has already experienced this transient before, he/she is required to identify its label. (430, 440). Bio signal and associated metadata segments from the occurrence are parsed and logged into the data set (450) under the respective mental state and transition labels. Else, the wearer is required to declare it as a new type of transition and give a new label (431, 441). A new label is created for the transition, data segments corresponding to the binary (initial and final) mental states are extracted from the bio and meta data channels, labeled appropriately, and added (450) to the data set.

FIG. 5 further explains process steps 311, 321 and 330 marked in FIG. 3 where the device detects transitions in the wearer's mind during the customization phase. It also represents a reinforcement learning process where in each pass of the cycle, the device is taught to pick (trigger on) only those transitions that its wearer is interested in. Every transition detected by the device gets filtered through the process leaving the most validated, reliable, and repeatable ones that would in turn be added to the validation data set and used to modify the PRM. Block 500 shows the step of the device getting triggered based on the latest version of the PRM (501) that it has access to. This would mean that the device would trigger on the transition labels manually added (as in FIG. 4) and/or the generic ones it inherited from the initial PRM model. Upon each triggering, the device saves the transition bio signal data along with relevant metadata and tries to match (520) them to that of the stored labels in its memory. If the device finds a match, the wearer is asked (530) if he/she agreed with the label the device found. If the wearer agrees with the finding (531) data segments corresponding to the constituent binary mental states are extracted from transition data (time series segments of bio signals and metadata collected from each data acquisition channel) and added to the validated dataset used to modify PRM (533). In case the wearer didn't agree with the match and thought that there was a better match with another label previously defined, he/she is given a chance to switch the label and add it to the validation data set (535). The wearer could also define it as a new type of transition, and label it appropriately (536) before using it for validation. In case the device can't find a match for the transition (540), it gives a chance to the wearer to start a new label (536) or identify it with an existing label (543). In either branch of the decision-making tree, the wearer is also given the option to mark the transition data as failed data (534) if no other options exist. Another occasion for failing the transition data (544), is when the device detects a transition, but the wearer didn't (512). After the device closes the interactive session at the end of each iteration (550) or a multiple of iterations, the resulting validation dataset could be used to re-train the PRM model 501 that was started with. These interactive sessions are implemented in a user-friendly manner, requiring a minimal number of screen touch/vocal command interactions and assisted by auditory instructions.

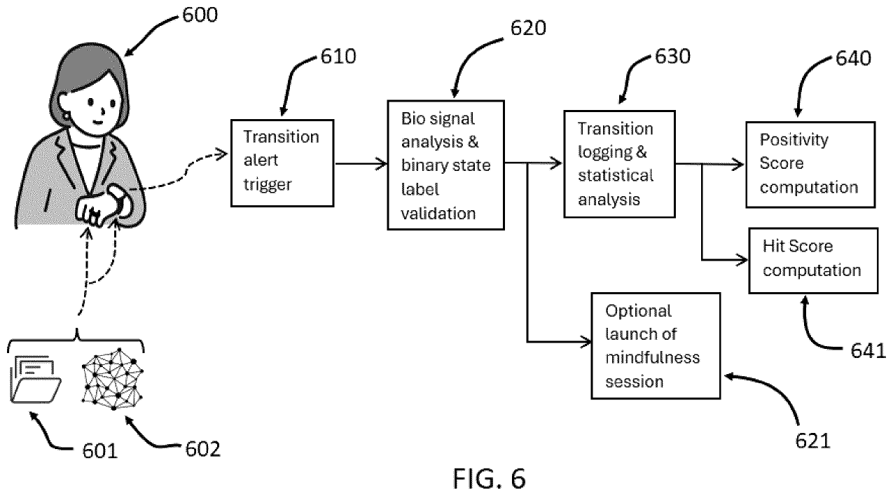

As the PRM matures, the device gets its full operational capabilities. FIG. 6 shows the typical action cycle of the device where a customized PRM 602 and a customized mantra library 601 (a set of wearer's favorite mantras) support the wearer 600 in daily life. Upon each transition alert trigger 610 caused by the bio signals and meta parameters (if factored in the PRM and if their sensors exist) crossing thresholds, wearer gets an opportunity to reflect on himself/herself increasing his/her mindfulness in day-to-day life. Further, transition labels are validated (620) and events are logged (630) for statistical analysis and computation of the wearer's performance scores, such as a Positivity Score (PS) and a Hit Score (HS). PS computed by 640, is a percentage fraction of the total dwelling time of the wearer's mind in a ‘positive state’ which is one of the pair of binary mental states used to build the PRM, chosen by the wearer to represent ‘positivity’. A positive mental state is achieved, mostly by using appropriate mantras for alerts and staying positive from its after-effects. The percentage fraction could be based off the total wakeful time in a day or a work-week of the wearer. HS is a metric computed 641, showing the total number of transition alerts played in a day which indicates the number of successful hits or interventions that the device performed in a day. A low HS would ideally mean that the device wasn't presented with enough occurrences of the designated transitions (labels) perhaps because of its rarity under the current circumstances of the wearer's life. If desired, the wearer could increase the HS by either trying a new type (label) or adding new labels to the device. An increased HS would mean more chances for the wearer to be self-aware of emotions and be self-reflecting that way. It may be noted in that a high Hit Score wouldn't necessarily imply a high value for PS. Results from 630, 640 and 641 are used to update the display depicted in FIGS. 2A & 2B in real time.

Block 621 depicts the possibility of launching non-blocking mindfulness sessions if desired by the wearer. The device can initiate a controlled breathing session guided by non-visual (audio and/or haptic) cues that wouldn't interfere with the wearers ongoing activities. Another capability of the device is of launching a ‘mindful walking session’ for the wearer, where he/she is cued to pace steps adaptively with his/her breathing cycles.

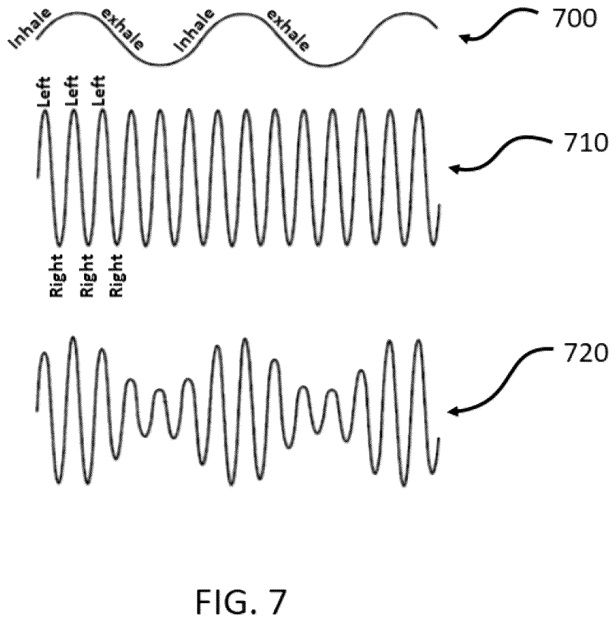

FIG. 7 shows three waveforms 700, 710 and 720 that represent rhythmic cues generated by the device to guide the wearer's breathing and/or walking. Cues are generated by modulating an attribute (such as amplitude, frequency, bias, periodicity etc. chosen depending on wearer's preference) of an audio tone or a tactile force. For the sake of simplicity, let's consider the use of a continuous audio tone of constant frequency for cue-generation purposes. Also, for simplicity, let us assume the expansion and contraction of lungs to follow a sinusoidal pattern during the inhalation and exhalation processes. (In practice, those patterns could be much more complicated with multiple amplitude and phase asymmetries, nonlinearities, kinks etc.). Modulations shown in FIG. 7 are sinusoids indicating sinusoidal variations in the amplitude of the previously mentioned audio tone of constant frequency. All modulated waveforms of FIG. 7 could be continuously generated using arbitrary waveform generators (AWGs) and phase/amplitude modulators. 700 represents an audio tone mimicking the inhalation and exhalation sounds (or expansion and contraction of lungs) of a person that repeats rhythmically in a sine wave pattern. In the actual working of the device, intensity of the tone is set to modulate (for example, increasing throughout the inhale period, pausing for a moment, decreasing throughout the exhale period and again pausing for a moment) in tandem with the instantaneous phase of a pre-defined breathing pattern, so that a device-wearer hearing the tone would be able to easily pace his/her breathing in phase-synchrony with the tone's intensity. The tone could also be a mix of frequencies or a musically composed piece designed to the listening pleasure of the wearer. In lieu of a tone, it could even be the voice of a person counting up or down through the breath cycles for pre-defined number of counts. Breathing pace or rhythm formulated by the device for the wearer could be one of the many ‘calm breathing patterns’ that the wearer could choose from or, an adaptive pattern that adjusts cycles in accordance with his/her breathing behavior and addressing anomalies such as tiredness, rapidity, shallowness, gasping etc. on the run. In the latter case, the device monitors breathing movements of the wearer through its sensors. Waveform 710 represents a pattern of rhythmic audio pulses (marked as ‘Left’ and ‘Right’ on 710) generated by the device in phase synchrony with the breadth-modulation cycles 700 (described above), in order to mimic the left and right strides (paces or steps) of a leisurely walking person. In this example case, waveform 710 would be generated by ON/OFF modulating another audio tone of the wearer's liking. The device could also adaptively pace the pulses according to the wearer's condition (such as energy, enthusiasm, tiredness) using feedback from its sensors. The pulses may be musically engineered bursts, cue word pronunciations (such as ‘Left’ and ‘Right’) etc. and could also skip alternate pulses intentionally for convenience, whichever way the wearer may desire. In many cases the wearer would want to hear an overlay of the two rhythms (700 and 710) in order to pace their walking steps in synchrony with the breathing cycle. In that case, waveforms 700 and 710 are initialized, added and streamed phase-synchronously to the ears of the wearer.

However, some other wearers might feel that an amplitude-modulated version of carrier 710 using the modulator 700 would be more musical to their ears. For those, the device produces waveform 720 which is essentially a foot stepping rhythm of pre-defined periodicity, rising and falling in intensity gradually in accordance with the instantaneous phase of a pre-defined sequence of inhalation and exhalation periods. A simple “mindful walking” session would involve an equal number of walking strides (Left-Right cycles of 710) under each of the inhalation and exhalation periods which are set equal as shown in 700. However, the wearer has the option to change the number of strides under inhale or exhale phases independently if needed.

I claim:

1. A wearable device comprising:
a) a reporting mean to self-report transitions in at least a first of multiple types of mental states happening in said wearer's mind, where activation of said reporting means is timed substantially in tandem with said transitions happening in said wearer's mind;
b) at least one data acquisition channel configured to acquire at least one type of bio signal from said wearer's body suiting to said at least a first type of mental state;
c) where said transition in mental state comprise of shifting of said wearer's mind from an initial state to a final state that together form a pair of binary mental states;
d) where said data acquisition channel upon activation of said reporting means is configured to trigger collection of at least one segment of said at least one type of bio signal from said wearer's body corresponding to the duration of said transition of mental state; and
e) software and hardware means programmed to parse signal segments corresponding said initial state and said final state of said wearer's mind from said at least one segment of said one type of bio signal.

2. Wearable device of claim 1 where said reporting mean comprise of at least one user interface panel carrying at least one readily accessible and prominent boolean button configured to receive a touch input.

3. Wearable device of claim 1 where said reporting mean comprise of at least one sensor configured to receive at least one of, but not limited to a voice command, a shake input or a hand gesture input.

4. Wearable device of claim 1 having at least one additional data acquisition channel and additional software and hardware means:
a) where said additional data acquisition channel upon activation of said reporting mean is configured to trigger collection of at least one segment of meta data from said wearer's environment and corresponding to the duration of said transition of mental state; and
b) said software and hardware means configured to parse segments corresponding said initial state and said final state from said at least one segment of meta data.

5. Said reporting mean of claim 1 having at least one user interface panel carrying two readily accessible, prominent boolean buttons where activation of one of said boolean buttons is programmed to convey said wearer's pick from a set of binary choices.

6. Wearable device of claim 1 handling transitions in said multiple types of mental states comprising:
a) at least one reporting mean for self-reporting transitions in each of said multiple types of mental states of said wearer;
b) at least one data acquisition channel configured to acquire at least one type of bio signal from said wearer's body corresponding to each of said multiple types of mental states; and
c) software and hardware means programmed to parse signal segments corresponding said initial state and said final state of said wearer's mind belonging to each of said multiple types of mental states.

7. Claim 1 where said reporting mean is remotely and communicatively coupled to body of said wearable device using wireless means.

8. Wearable device of claim 1 additionally comprising a readily accessible, quick-glance display panel programmed to comparingly display dwelling periods of said wearer's mind in either one of a pair of binary mental states, belonging to at least a said first of multiple types of mental states in real time:
a) where said display panel has a clock face having a time scale marked on a closed curve;
b) where said clock face has a time-hand that indicates time and a said binary mental state where said wearer's mind is dwelling; and
c) where said display of dwelling periods comprise of circular or annular pie charts whose arc lengths represent durations of said wearer's mind staying in said either one of a pair of binary mental states.

9. A device programmed to comparingly display dwelling periods of a person's mind in a pair of binary mental states,
a) where said display of dwelling periods is a clock face having a time scale marked on a closed curve; and
b) where said display of dwelling periods comprise of circular or annular pie charts whose arc lengths represent durations of said wearer's mind staying in said either one of said pair of binary mental states.

10. A wearable device having at least one non-visual cueing means to conduct a mindful breathing session comprising:
a) Means to generate at least one breathing pattern suiting said wearer's preferences;
b) Means to modulate at least one attribute of at least one of a tactile force or an audio tone in accordance with instantaneous phase of said breathing pattern in repeating cycles;
c) Said at least one attribute belonging but not limited to amplitude, frequency, periodicity and bias; and
d) Means to communicate modulated cycles of said at least one attribute of said at least one of a tactile force or an audio tone to said wearer as a cue for breathing.

11. Device of claim 10 where said device has additional means to adaptively pace said cue for breathing according to said wearer's physical conditions.

12. Device of claim 10 where said cueing means having additional means to generate, modulate and communicate a cue for pacing footsteps in phase-synchrony with said modulated cycles of said at least one attribute of said at least one of a tactile force or an audio tone to said wearer.

13. Device of claim 10 where said device has additional means to adaptively pace said cue for breathing and said cue for pacing footsteps according to said wearer's physical conditions.

14. Method of building a Physiological Response Model PRM of a person, capable of computing a binary response output representing either one of a pair of binary mental states of said person undergoing a mental state transition, from signal segments collected from said person's body in tandem with said person's mind transitioning from a first to a second of said pair of binary mental states, comprising:
a) a method of having said person acknowledge said mental state transition and label it for said device via user inputs;
b) a method of having said person acknowledge at least a first of a newly felt mental state transition, compare said newly felt mental state transition against said labeled mental state transition of 14(a) for its constituent binary mental states and report a case of matching or non-matching between the two mental state transitions for said device via said user inputs;
c) a method of having said device extract one said signal segment from said person's body corresponding to each of said pair of binary mental states of said newly felt mental state transition of said person; and
d) a method of using said extracted signal segments of 14(c) and said matching or non-matching report of 14(b) corresponding to each of said newly felt mental state transitions of said person, to create a PRM of said person by means not limited to training of an artificial neural network.

15. Method 14 of building a Physiological Response Model PRM of a person:
a) where method of 14(c) having said device to additionally extract said signal segment from said person's environment corresponding to each of said binary mental states of said newly felt mental transition of said person; and
b) where method 14(d) of using said extracted signal segments of 14(c) and said matching or non-matching report of 14(b), additionally using said additionally extracted signal segments of 15(a) corresponding to each of said newly felt mental transitions, to create said PRM of said person by means not limited to said training of an artificial neural network.

16. A method of using at least one non-visual cueing means to conduct a mindful breathing session comprising:
a) a method of generating at least one breathing pattern mimicking the rise and fall of airflow during inhalation and exhalation;
b) a method of breathe-modulating at least one attribute such as, but not limited to an amplitude, frequency, periodicity and bias of at least one of a tactile force or a first audio tone in accordance with instantaneous phase of said breathing pattern in repeating cycles; and
c) a method of communicating said breathe-modulated cycles of said at least one attribute of said at least one of a tactile force or a first audio tone to said wearer as a cue for breathing.

17. Method of 16 where said using of non-visual cueing means additionally include a method to generate, modulate and communicate a cue for pacing footsteps in phase-synchrony with, said breathe-modulated cycles, by modulating said at least one attribute of said at least one of a tactile force or a second audio tone, to said wearer.
